SEQUENCE LISTING (31 sequences)

SEQ ID NO: 1 - protein, 118 aa, synthetic construct
QVTLKESGPALVKPTQTLTLTCTFSGFSLSTFAMGVGWIRQPPGKGLEWIAHIWWDDDKYYNSVLKSRLTISKDTSKNQVVLTMTNMDPVDTATYYCARLGGYAMDYWGQGTLVTVSS

SEQ ID NO: 2 - protein, 107 aa, synthetic construct
DIQMTQSPSSLSASVGDRVTITCRASQDIGNNLNWLQQKPDGTIKRLIFATSRLDSGVPSRFSGSGSGTEFTLTISSLQPEDFATYYCLQYASSPYTFGGGTKVEIK

SEQ ID NO: 3 - protein, 7 aa, synthetic construct
TFAMGVG

SEQ ID NO: 4 - protein, 16 aa, synthetic construct
HIWWDDDKYYNSVLKS

SEQ ID NO: 5 - protein, 8 aa, synthetic construct
LGGYAMDY

SEQ ID NO: 6 - protein, 11 aa, synthetic construct
RASQDIGNNLN

SEQ ID NO: 7 - protein, 7 aa, synthetic construct
ATSRLDS

SEQ ID NO: 8 - protein, 9 aa, synthetic construct
LQYASSPYT

SEQ ID NO: 9 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 10 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 11 - protein, 5 aa, synthetic construct
RYTMH

SEQ ID NO: 12 - protein, 17 aa, synthetic construct
YINPSRGYTNYNQKFKD

SEQ ID NO: 13 - protein, 10 aa, synthetic construct
YYDDHYCLDY

SEQ ID NO: 14 - protein, 10 aa, synthetic construct
RASSSVSYMN

SEQ ID NO: 15 - protein, 7 aa, synthetic construct
DTSKVAS

SEQ ID NO: 16 - protein, 9 aa, synthetic construct
QQWSSNPLT

SEQ ID NO: 17 - protein, 107 aa, synthetic construct [sequence omitted]

SEQ ID NO: 18 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLASRLTVDKSRWQEGNVFSCSVMHEALHNHYTQKSLSLSLGK

SEQ ID NO: 19 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 10 aa, synthetic construct
GGGGSGGGGS

SEQ ID NO: 21 - protein, 214 aa, synthetic construct
DIQMTQSPSSLSASVGDRVTITCRASQDIGNNLNWLQQKPDGTIKRLIFATSRLDSGVPSRFSGSGSGTEFTLTISSLQPEDFATYYCLQYASSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 445 aa, synthetic construct
QVTLKESGPALVKPTQTLTLTCTFSGFSLSTFAMGVGWIRQPPGKGLEWIAHIWWDDDKYYNSVLKSRLTISKDTSKNQVVLTMTNMDPVDTATYYCARLGGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 698 aa, synthetic construct
QVTLKESGPALVKPTQTLTLTCTFSGFSLSTFAMGVGWIRQPPGKGLEWIAHIWWDDDKYYNSVLKSRLTISKDTSKNQVVLTMTNMDPVDTATYYCARLGGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLASRLTVDKSRWQEGNVFSCSVMHEALHNHYTQKSLSLSLGK

SEQ ID NO: 24 - protein, 659 aa, synthetic construct
QVTLKESGPALVKPTQTLTLTCTFSGFSLSTFAMGVGWIRQPPGKGLEWIAHIWWDDDKYYNSVLKSRLTISKDTSKNQVVLTMTNMDPVDTATYYCARLGGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLASRLTVDKSRWQEGNVFSCSVMHEALHNHYTQKSLSLSLGK

SEQ ID NO: 25 - protein, 226 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 433 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLASRLTVDKSRWQEGNVFSCSVMHEALHNHYTQKSLSLSLGK

SEQ ID NO: 27 - protein, 330 aa, synthetic construct [sequence omitted]

SEQ ID NO: 28 - protein, 107 aa, synthetic construct [sequence omitted]

SEQ ID NO: 29 - protein, 440 aa, synthetic construct
QVQLVQSGAEVKKPGASVTVSCKASGYTFTDYEMHWIRQPPGEGLEWIGAIDGPTPDTAYSEKFKGRVTLTADKSTSTAYMELSSLTSEDTAVYYCTRFYSYTYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 30 - protein, 447 aa, synthetic construct
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFASTYRVVSSLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSSGSFFLYSKLTVDKSRWQEGNVFSCSVMHEALHNHYTQKSLSLSP

SEQ ID NO: 31 - protein, 219 aa, synthetic construct
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0347492 A1
(43) Pub. Date: Nov. 5, 2026

(54) BISPECIFIC ANTIBODY BINDING TO BAFFR AND CD3 AND USE THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Liusong YIN, Nanjing (CN); Xiaoling JIANG, Nanjing (CN)

(73) Assignee: SHENGHE (CHINA) BIOPHARMACEUTICAL CO., LTD., Nanjing, Jiangsu (CN)

(21) Appl. No.: 19/118,521

(22) PCT Filed: Oct. 8, 2023
(86) PCT No.: PCT/CN2023/123288

§ 371 Date: Apr. 4, 2025

(30) Foreign Application Priority Data
Oct. 8, 2022 (WO) PCT/CN2022/123744

(51) Int. Cl. C07K 16/28; A61K 38/00; A61K 47/68

(52) U.S. Cl. CPC C07K 16/2878; A61K 38/00; A61K 47/6849; C07K 16/2809; C07K 2317/31; C07K 2317/35; C07K 2317/52; C07K 2317/55; C07K 2317/565; C07K 2317/76

(57) ABSTRACT

A bispecific antibody, including: (a) a first antigen-binding portion that specifically binds to the first antigen, where the first antigen is BAFFR; (b) a second antigen-binding portion that specifically binds to the second antigen, where the second antigen is BAFFR; and (c) a third antigen-binding portion that specifically binds to the third antigen, where the third antigen is CD3. The bispecific antibody maximizes tumor killing effect by multivalently binding to tumor-associated antigens while controlling CD3 toxicity; the BAFFR antibody multivalently binds to the BAFFR-positive tumor, and the CD3 antibody at the other end binds to the T cell. The bispecific antibody acts as an adapter, bringing the T cell and the tumor cell closer to form an immune synapse, so that the T cell kills the tumor. The monovalent CD3 antibody reduces toxicity and can better form an immune synapse to achieve a more effective, specific, and safer anti-tumor response.

### TECHNICAL FIELD

The present invention belongs to the field of biomedicine and specifically relates to a bispecific antibody binding to BAFFR and CD3 and use thereof.

### BACKGROUND

CD3 (T-cell surface glycoprotein CD3, signal transduction co-receptor of the T-cell receptor, which contains subunits γ, δ, ε, and ζ) is a differentiation antigen expressed on the surface of all T-lymphocytes and primarily mediates the transduction of T-cell activation signals. It plays an important role in the anti-infection immunity of the body's immune system. CD3 molecules form a stable TCR-CD3 complex with T cell antigen receptors, whose extracellular region recognizes and binds major histocompatibility complex class II molecules and enhances the stability of the binding of T cell antigen receptors to MHC molecules, while the intracellular region enhances the activation signals transduced by leukocytes' CD3, thus participating in and regulating the activation of the immune system. The CD3-positive lymphocyte population is an important indicator of cellular immunity.

The only ligand of BAFFR is BAFF, in addition to being a marker for specific developmental stages of B cells, BAFFR binds to BAFF and acts together to activate the NF-kB signaling pathway in B cells to promote the proliferation and activation of B cells. The BAFF-BAFFR signaling pathway and the BCR pathway are key pathways for Immature B cell maturation and activation, and blockade of the dual pathways completely removes peripheral B lymphocytes and inhibits B lymphocyte development and proliferation.

Clinical studies show that about 30-60% of NHL patients receiving Mabthera for the first time on single-dose therapy are resistant, and about 60% of relapsed patients are resistant to a second single-dose treatment with Mabthera, and that the primary mechanism of resistance is either insufficient abundance of CD20 expression or shedding of CD20 from B lymphocytes. CD20 is not essential for B-cell growth, and in vitro studies show no change in the growth rate of B lymphocytes after knockdown of CD20.BAFFR is expressed in higher abundance than CD20 and is an essential signaling pathway protein for B-cell growth, which, together with the BCR pathway, is essential for B-cell proliferation and activation. In vivo pharmacodynamic assays show that the BAFFR-targeting antibody drugs C55 and C90 are as effective as Mabthera for the Mabthera-sensitive lymphoma cell Z138, completely removing lymphoma cells from the blood of mouse, and prolonging the tumor-free survival of mouse. For the Mabthera-resistant cell line (CD20 knockout Raji), C90 still completely cleared lymphoma cells from the blood of mouse and prolonged the tumor-free survival of mouse to 100 days (within 20 days of Mabthera survival).

Therefore, as a downstream signaling pathway of BCR, BAFFR-targeted antibody can effectively address the ineffectiveness and relapse resistance of ibrutinib. However, the antitumor activity of ADCC activity of monoclonal antibody drugs is limited, and T cells as effective antitumor cells have been validated as an effective strategy, so the development of T-cell articulator therapies that target both T-cell receptors and tumor-associated antigens (TAA) has been widely used. Anti-CD3 and anti-BAFFR bispecific antibody can activate endogenous T cells by bispecific binding to BAFFR on the surface of tumor cells and CD3 on the surface of T cells, leading to targeted lysis of BAFFR-positive tumor cells.

### SUMMARY

In the present application, the inventors have developed a bispecific antibody with good performance capable of binding to BAFFR and CD3. The bispecific antibody of the present invention maximizes tumor killing effect by multivalently binding to tumor-associated antigens while controlling CD3 toxicity: the BAFFR antibody multivalently binds to the BAFFR-positive tumor, and the CD3 antibody at the other end binds to the T cell. The bispecific antibody acts as an adapter, bringing the T cell and the tumor cell closer to form an immune synapse, so that the T cell kills the tumor. The monovalent CD3 antibody reduces toxicity and can better form an immune synapse to achieve a more effective, more specific, and safer anti-tumor response.

The present invention provides a bispecific antibody, comprising: (a) a first antigen-binding portion that specifically binds to the first antigen, wherein the first antigen is BAFFR; (b) a second antigen-binding portion that specifically binds to the second antigen, wherein the second antigen is BAFFR; and (c) a third antigen-binding portion that specifically binds to the third antigen, wherein the third antigen is CD3.

In some embodiments, the first antigen-binding portion is a full-length antibody consisting of two heavy chains and two light chains.

In some embodiments, the second antigen-binding portion is an antibody fragment comprising a heavy chain variable domain (VH) and/or a light chain variable domain (VL).

In some embodiments, the third antigen-binding portion is an antibody fragment comprising a heavy chain variable domain (VH) and/or a light chain variable domain (VL).

In some embodiments, the second antigen-binding portion is Fab, Fab′, scFab, F(ab′)2, Fv, dsFv, or scFv. Preferably, the second antigen-binding portion is Fab.

In some embodiments, the third antigen-binding portion is Fab, Fab′, scFab, F(ab′)2, Fv, dsFv or scFv. Preferably, the third antigen-binding portion is scFv.

In some embodiments, the second antigen-binding portion is fused to the N-terminus of one heavy chain of the first antigen-binding portion.

In some embodiments, the second antigen-binding portion is fused to the C-terminus of one heavy chain of the first antigen-binding portion.

In some embodiments, the second antigen-binding portion is fused to the N-terminus and C-terminus of one heavy chain of the first antigen-binding portion.

In some embodiments, the second antigen-binding portion is fused to the N-terminus of both heavy chains of the first antigen-binding portion.

In some embodiments, the second antigen-binding portion is fused to the C-terminus of both heavy chains of the first antigen-binding portion.

In some embodiments, the second antigen-binding portion is fused to the N-terminus and the C-terminus of both heavy chains of the first antigen-binding portion.

In some embodiments, the second antigen-binding portion is fused to the N-terminus of one light chain of the first antigen-binding portion.

In some embodiments, the second antigen-binding portion is fused to the C-terminus of one light chain of the first antigen-binding portion.

In some embodiments, the second antigen-binding portion is fused to the N-terminus and the C-terminus of one light chain of the first antigen-binding portion.

In some embodiments, the second antigen-binding portion is fused to the N-terminus of both light chains of the first antigen-binding portion.

In some embodiments, the second antigen-binding portion is fused to the C-terminus of both light chains of the first antigen-binding portion.

In some embodiments, the second antigen-binding portion is fused to the N-terminus and the C-terminus of both light chains of the first antigen-binding portion.

In some embodiments, the third antigen-binding portion substitutes one or two of Fab regions of the first antigen-binding portion. Preferably, the third antigen-binding portion substitutes the Fab region of the first antigen-binding portion fused to the second antigen-binding portion.

In some embodiments, the third antigen-binding portion substitutes one or two of Fv regions of the first antigen-binding portion. Preferably, the third antigen-binding portion substitutes the Fv region of the first antigen-binding portion fused to the second antigen-binding portion.

In some embodiments, the second antigen-binding portion is fused to the N-terminus of one heavy chain of the first antigen-binding portion, and the third antigen-binding portion substitutes the Fab region of the first antigen-binding portion fused to the second antigen-binding portion.

In some embodiments, the second antigen-binding portion is fused to the N-terminus of one heavy chain of the first antigen-binding portion, and the third antigen-binding portion substitutes the Fv region of the first antigen-binding portion fused to the second antigen-binding portion.

In some embodiments, the bispecific antibody comprises a first Fc region and a second Fc region. Preferably, the first Fc region and the second Fc region are the same or different. Preferably, the Fc region is selected from IgG, IgA, IgD, IgE, IgM and variants thereof. Preferably, the Fc region is selected from IgG1, IgG2, IgG3, IgG4 and variants thereof.

In some embodiments, the Fc region comprises one or more amino acid mutations, preferably amino acid substitutions, insertions or deletions.

In some embodiments, the first Fc region is knob-Fc and the second Fc region is hole-Fc.

In some embodiments, the first Fc region is hole-Fc and the second Fc region is knob-Fc.

In some embodiments, the VH and VL of the third antigen-binding portion are interchanged.

In some embodiments, the second antigen-binding portion is fused to the N-terminus of one heavy chain of the first antigen-binding portion, the third antigen-binding portion substitutes the Fab region of the first antigen-binding portion fused to the second antigen-binding portion, and the VH and VL of the third antigen-binding portion are interchanged.

In some embodiments, the second antigen-binding portion is fused to the N-terminus of one heavy chain of the first antigen-binding portion, the third antigen-binding portion substitutes the Fv region of the first antigen-binding portion fused to the second antigen-binding portion, and the VH and VL of the third antigen-binding portion are interchanged.

In some embodiments, the second antigen-binding portion and the third antigen-binding portion are fused to the first antigen-binding portion by a linker.

In some embodiments, the linker is a peptide linker. Preferably, the peptide linker is a GS linker or a mutant human IgG hinge. Preferably, the peptide linker has an amino acid sequence as shown in (G4S)ₓ, x is an integer selected from 1-6; preferably, the peptide linker is (G4S)₂, (G4S)₃ or (G4S)₄. More preferably, the peptide linker is (G4S)₂.

In some embodiments, the first antigen-binding portion specifically binds to BAFFR, wherein HCDR1 of the first antigen-binding portion is shown in SEQ ID NO: 3, or is a sequence having at least 80% identity to SEQ ID NO: 3; HCDR2 is shown in SEQ ID NO: 4, or is a sequence having at least 80% identity to SEQ ID NO: 4; HCDR3 is shown in SEQ ID NO: 5, or is a sequence having at least 80% identity to SEQ ID NO: 5; LCDR1 is shown in SEQ ID NO: 6, or is a sequence having at least 80% identity to SEQ ID NO: 6; LCDR2 is shown in SEQ ID NO: 7, or is a sequence having at least 80% identity to SEQ ID NO: 7; and LCDR3 is shown in SEQ ID NO: 8, or is a sequence having at least 80% identity to SEQ ID NO: 8.

In some embodiments, the second antigen-binding portion specifically binds to BAFFR, wherein HCDR1 of the second antigen-binding portion is shown in SEQ ID NO: 3, or is a sequence having at least 80% identity to SEQ ID NO: 3; HCDR2 is shown in SEQ ID NO: 4, or is a sequence having at least 80% identity to SEQ ID NO: 4; HCDR3 is shown in SEQ ID NO: 5, or is a sequence having at least 80% identity to SEQ ID NO: 5; LCDR1 is shown in SEQ ID NO: 6, or is a sequence having at least 80% identity to SEQ ID NO: 6; LCDR2 is shown in SEQ ID NO: 7, or is a sequence having at least 80% identity to SEQ ID NO: 7; and LCDR3 is shown in SEQ ID NO: 8, or is a sequence having at least 80% identity to SEQ ID NO: 8.

In some embodiments, the third antigen-binding portion specifically binds to CD3, wherein HCDR1 of the third antigen-binding portion is shown in SEQ ID NO: 11, or is a sequence having at least 80% identity to SEQ ID NO: 11; HCDR2 is shown in SEQ ID NO: 12, or is a sequence having at least 80% identity to SEQ ID NO: 12; HCDR3 is shown in SEQ ID NO: 13, or is a sequence having at least 80% identity to SEQ ID NO: 13; LCDR1 is shown in SEQ ID NO: 14, or is a sequence having at least 80% identity to SEQ ID NO: 14; LCDR2 is shown in SEQ ID NO: 15, or is a sequence having at least 80% identity to SEQ ID NO: 15; and LCDR3 is shown in SEQ ID NO: 16, or is a sequence having at least 80% identity to SEQ ID NO: 16.

In some embodiments, the first antigen-binding portion specifically binds to BAFFR, wherein the heavy chain variable region VH of the first antigen-binding portion is as shown in SEQ ID NO: 1, or is a sequence having at least 80% identity to SEQ ID NO: 1; and the light chain variable region VL is as shown in SEQ ID NO: 2, or is a sequence having at least 80% identity to SEQ ID NO: 2.

In some embodiments, the second antigen-binding portion specifically binds to BAFFR, wherein the heavy chain variable region VH of the second antigen-binding portion is as shown in SEQ ID NO: 1, or is a sequence having at least 80% identity to SEQ ID NO: 1; and the light chain variable region VL is as shown in SEQ ID NO: 2, or is a sequence having at least 80% identity to SEQ ID NO: 2.

In some embodiments, the third antigen-binding portion specifically binds to CD3, wherein the heavy chain variable region VH of the third antigen-binding portion is as shown in SEQ ID NO: 9, or is a sequence having at least 80% identity to SEQ ID NO: 9; and the light chain variable region VL is as shown in SEQ ID NO: 10, or is a sequence having at least 80% identity to SEQ ID NO: 10.

The present invention also provides an isolated nucleic acid molecule, comprising a nucleotide sequence encoding the bispecific antibody as described in any one of the above.

The present invention also provides use of the bispecific antibody described in any one of the above embodiments in the preparation of a drug for the treatment of cancer. In some embodiments, the cancer is selected from astrocytoblastoma of the human brain, human pharyngeal head cancer, adrenal gland tumors, AIDS-associated cancers, adenovascular soft tissue sarcomas, astrocytomas, bladder cancers, bone cancers, brain and spinal cord cancers, metastatic brain tumors, breast cancers, carotid body tumors, cervical cancers, chondrosarcoma, chordoma, renal smoky cell carcinoma, clear cell carcinoma, colon cancer, colorectal cancer, connective tissue-promoting proliferative small round cell tumors, ventricular meningiocytoma, Ewing's tumor, extraskeletal mucinous chondrosarcoma, osteofibrous dysplasia, fibrous dysplasia of the bone, carcinoma of the gallbladder or bile ducts, gastric cancer, gestational Trophoblastic Disease, Germ Cell Tumor, Head and Neck Cancer, Hepatocellular Carcinoma, Islet Cell Tumor, Kaposi's Sarcoma, Kidney Cancer, Leukemia, Liposarcoma/Malignant Lipomatous Tumor, Hepatocellular Carcinoma, Lymphoma, Lung Cancer, Adenodermatous Tubular Cell Tumor, Melanoma, Meningioma, Multiple Endocrine Neoplasia, Multiple Myeloma, Myelodysplastic syndromes, adult neuroblastoma, neuroendocrine tumors, ovarian cancer, pancreatic cancer, papillary thyroid cancer, parathyroid adenoma, pediatric cancers, peripheral nerve sheath tumors, pheochromocytomas, pituitary tumors, prostate cancers, posterior uveal melanoma, metastatic renal carcinoma, rhabdomyosarcoma, rhabdomyosarcoma, sarcoma, skin cancers, soft-tissue sarcoma, squamous-cell carcinoma, synovial sarcoma, testicular cancer, Thymic carcinoma, thymoma, Metastatic cancer of the thyroid gland or uterine cancer.

The present invention also provides use of the bispecific antibody described in any one of the above embodiments in the preparation of a drug for the treatment of an autoimmune disease. In some embodiments, the autoimmune disease is selected from graft-versus-host disease, rheumatoid arthritis, Crohn's disease, multiple sclerosis, colitis, psoriasis, autoimmune uveitis, aspergillosis, herpetic epidermolysis bullosa, or type I diabetes mellitus.

In some embodiments, the use is achieved by one or more of tumor immunotherapy, cell therapy, or gene therapy.

The present invention also provides a pharmaceutical composition, comprising the bispecific antibody as described in any one of the above and a pharmaceutically acceptable carrier, diluent or excipient.

The present invention also provides an antibody drug conjugate, comprising the bispecific antibody as described in any one of the above embodiments. In some embodiments, the coupling drug is selected from a cytotoxin, a small molecule chemical, or an immunotoxin.

### ABBREVIATIONS AND DEFINITIONS OF TERMS

The following abbreviations are used in this patent. VH: variable region of the heavy chain of the antibody; VL: variable region of the light chain of the antibody; CDR: complementary determining region in the variable region of the immunoglobulin; IgG: immunoglobulin G.

The term “antibody” refers to a natural immunoglobulin or an immunoglobulin prepared by partial or complete synthesis. The antibody may be isolated from a natural source such as plasma or serum in which the antibody is naturally present, or from the culture supernatant of antibody-producing hybridoma cells, or from the immune serum of an animal, or from reconstruction by phage library screening. Alternatively, they may be partially or completely synthesized by using techniques such as genetic recombination. Preferred antibodies include, for example, antibodies to isoforms of immunoglobulins or subclasses of these isoforms. Human immunoglobulins are known to include nine classes (isotypes) of IgG1, IgG2, IgG3, IgG4, IgA1, IgA2, IgD, IgE, and IgM. Within these isotypes, the antibodies of the present invention may include IgG1, IgG2, IgG3, and/or IgG4.

Some of the antibodies used herein consist of an immunoglobulin molecule comprising two pairs of polypeptide chains, each pair having a light chain (LC) and a heavy chain (HC). Each heavy chain consists of a heavy chain variable region (VH) and a heavy chain constant region (CH). The heavy chain constant region consists of three structural domains (CH1, CH2 and CH3). Each light chain consists of a light chain variable region (VL) and a light chain constant region (CL), or only a light chain constant region (CL). The light chain constant region consists of a structural domain CL. The constant structural domain is not directly involved in antibody-antigen binding, but exhibits a variety of effector functions, such as the ability to mediate the binding of immunoglobulins to host tissues or factors, including various cells of the immune system (e.g., effector cells) and the first component of the classical complement system (C1q). The VH and VL regions can also be subdivided into regions with high variability (called complementary determining regions (CDRs)), with more conserved regions called framing regions (FRs) scattered in between. Each VH and VL consists of three CDRs and four FRs arranged from amino-terminus to carboxy-terminus in the following order: FR1, CDR1, FR2, CDR2, FR3, CDR3, FR4. The variable regions of each heavy chain/light chain pair (VH and VL) form the antigen-binding site, respectively.

The term “antigen-binding portion” or “antigen-binding fragment” refers to one or more portions of an antibody that retain the ability to bind the antigen bound by the antibody. Examples of “antigen-binding fragments” of an antibody include (1) Fab fragments; (2) F(ab′)2 fragments; (3) Fd fragments, consisting of the VH and CH1 domains; (4) Fv fragments; (5) dAb fragments, consisting of the VH domains; and (6) CDRs, which have been isolated as complementary decision regions.

In addition, although the two structural domains of the Fv fragment, VL and VH, are encoded by separate genes, recombinant methods can be used to link them by synthetic linkers, thereby enabling the production of a single protein chain for which the VL and VH regions are paired to form a monovalent molecule (referred to as single-chain Fv (scFv)). Such single-chain antibody is also included in the term “antigen-binding fragment” of an antibody. Such antibody fragments are obtained using conventional techniques known to those skilled in the art, and the fragments are screened for functionality in the same manner as for intact antibodies. The antigen-binding portion may be produced by recombinant DNA techniques or by enzymatic or chemical cleavage of the intact immunoglobulin. The antigen-binding fragment may also be incorporated into a single-chain molecule comprising a pair of tandem Fv fragments (VH-CH1-VH-CH1) which, together with a complementary light chain polypeptide, form a pair of antigen-binding regions.

The term “Fab fragment” consists of the intact L chain, the variable region structural domain (VH) of the H chain and the first constant domain (CH1) of a heavy chain. Each Fab fragment is monovalent for antigen binding, i.e., it has a single antigen binding site. For example, Fab fragments can be produced recombinantly or by papain digestion of full-length antibody.

The term “Fab′ fragment” differs from Fab fragments in that several additional residues are added to the carboxyl terminus of the CH1 domain, including one or more cysteines from the hinge region of the antibody.Fab′ can be produced by treating F(ab′)2, which specifically recognizes and binds to the antigen, with a reducing agent such as dithiothreitol.

The term “F(ab′)2 fragment” was originally created as a pair of Fab′ fragments with hinge cysteines in between. The F(ab′)2 fragments can be produced recombinantly or by pepsin digestion of intact antibody (which removes most of the Fc region while retaining portions of the intact hinge region). The F(ab′)2 fragment can be dissociated (into two Fab′ molecules) by treatment with a reducing agent such as β-mercaptoethanol.

The term “scFab” refers to a single-chain Fab fragment, which is formed by introducing a polypeptide linker between the heavy chain variable domain (VH) and the light chain (CL) to form a single-chain Fab fragment (scFab).

The term “Fv” refers to the smallest antibody fragment that contains the complete antigen recognition and binding site. The fragment consists of a dimer formed by tight non-covalent binding of a heavy chain variable region structural domain and a light chain variable region structural domain. Folding of these two domains produces six highly variable loops (three loops from the H chain and three loops from the L chain) that contribute amino acid residues for antigen binding and confer antigen binding specificity to the antibody. However, even though the individual variable domains have the ability to recognize and bind antigen, their affinity is low compared to the full binding site.

The term “scFv” fragment refers to an antibody fragment comprising the VH and VL structural domains of the antibody, wherein these structural domains are present in a single polypeptide chain, the Fv polypeptide can further comprise a polypeptide linker between the VH and VL structural domains, wherein the polypeptide linker enables the scFv to form the desired structure for antigen binding. The “scFv-Fc” fragment comprises scFv linked to the Fc structural domain. e.g., the Fc structural domain may be linked to the C-terminus of scFv. Depending on the orientation of the variable structural domain in the scFv (i.e., VH-VL or VL-VH), the Fc structural domain can be after VH or VL. the Fc structural domain can be any suitable Fc structural domain known in the art or described herein. In some embodiments, the Fc structural domain is an IgG1Fc structural domain.

The term “dsFv” refers to disulfide bond stabilized Fv fragments. In dsFv, polypeptides in which one amino acid residue in each VH and VL is replaced by a cysteine residue are linked by a disulfide bond between the cysteine residues. To produce such molecules, one amino acid in the framework region of each of VH and VL is mutated to a cysteine, which in turn forms a stable interchain disulfide bond. Typically, position 44 in VH and position 100 in VL are mutated to cysteines. The term dsFv encompasses both dsFv (molecules in which VH and VL are linked by an interchain disulfide bond rather than a linker peptide) or scdsFv (molecules in which VH and VL are linked by both a linker and an interchain disulfide bond), as known in the art.

The term “epitope” refers to an antigenic determinant cluster in an antigen and the antigenic site bound by the structural domain of an antigen-binding molecule comprising an antibody variable region as disclosed in this specification. Thus, the epitope can be defined based on its structure. In addition, the epitope may also be defined based on the antigen-binding activity in the antigen-binding molecule that recognizes the epitope. When the antigen is a peptide or polypeptide, the epitope may be designated by the amino acid residues forming the epitope; when the epitope is a glycan chain, the epitope may be defined by its specific glycan chain structure.

The term “specificity” refers to the fact that one of the molecules involved in specific binding does not show any significant binding to a molecule or molecules different from one or more of the binding partner molecules. In addition, the term is used when the structural domain containing the variable region of the antibody is specific to a particular epitope of a plurality of epitopes in the antigen. When the epitope to which the domain containing the antibody variable region binds is contained in several different antigens, an antigen-binding molecule comprising the domain containing the antibody variable region can bind a variety of antigens having the epitopes.

The term “bispecific antibody” refers to a protein molecule capable of binding specifically to two target antigens or target antigen epitopes. In the present invention, “bispecific antigen-binding protein” comprising an antibody or antigen-binding fragment (e.g., Fab, scFv, etc.) is used interchangeably with “bispecific antibody”, and “bis-antibody”.

The term “knob-Fc” refers to the replacement of an amino acid residue in the CH3 domain of the first subunit of the Fc structural domain with an amino acid residue having a larger side chain volume, thereby creating a bulge in the CH3 domain of the first subunit that can be localized in a depression in the CH3 domain of the second subunit. For example, by mutating a serine T at position 366 of CH3 of a heavy chain to a tryptophan W, a protruding “knob”-like bulge is formed.

The term “hole-Fc” refers to the replacement of an amino acid residue in the CH3 domain of the second subunit of the Fc structural domain with an amino acid residue having a smaller side chain volume, thereby creating a depression in the CH3 domain of the second subunit within which a bulge in the CH3 domain of the first subunit can be positioned. For example, by mutating a serine T at position 366 to serine S, a leucine L at position 368 to alanine A, and an amino acid at position 407 from tyrosine Y to valine V or to alanine A of an additional heavy chain, the mutation creates a concave “hole”-like depression.

The term “fusion” refers to the linking of two amino acid sequences to form a new sequence, e.g., by means of a linker, to form a new synthetic protein or antibody.

The term “linker” or “L1” used to connect two protein domains refers to a connectivity peptide sequence that is used to connect protein domains and has a certain degree of flexibility. The use of a linker does not cause the loss of the original function of the protein domain.

The term “variable region” or “variable domain” of an antibody refers to the variable region of the light chain (VL) of an antibody or the variable region of the heavy chain (VH) of an antibody, individually or in combination. As known in the art, the variable regions of the heavy chain and the light chain each comprise four framework regions (FRs) linked by three complementary decision regions (CDRs) (also known as highly variable regions). The CDRs in each chain are held tightly together by the FRs and together with the CDRs from the other chain contribute to the formation of the antigen-binding site of the antibody.

The term “variable” refers to the fact that certain segments of the variable domains vary widely in sequence from antibody to antibody.V structural domains mediate antigen binding and define the specificity of a particular antibody for its particular antigen. However, variability is not uniformly distributed over the entire range of variable domains. Instead, it is concentrated in three segments within the light- and heavy-chain variable domains known as highly variable regions (HVRs). The more highly conserved portion of the variable domain is called the framework region (FR). The variable domains of the natural heavy and light chains each contain four FR regions, most of which adopt a β-folded conformation and are connected by three HVRs, which form a ring connection and, in some cases, form part of the β-folded structure. The HVRs in each chain are closely held together by the FR regions and, together with the HVRs of the other chains, contribute to the formation of the antigen-binding site of the antibody. The HVRs are not directly implicated in antibody-antigen binding, but exhibit various effector functions, such as involvement in antibody-dependent cytotoxicity of antibody.

The term “antibody-drug conjugate” or “ADC” refers to a binding protein (e.g., an antibody or antigen-binding fragment thereof) that is coupled to one or more coupled drugs (which may optionally be therapeutic or cytotoxic agents), and whose structure typically consists of three parts: the antibody or antibody-like ligand, the drug portion, and a linker that couples the antibody or antibody-like ligand and the drug. An ADC typically has 1, 2, 3, 4, 5, 6, 7, 8, 9, or 10 numbers of drugs coupled to the antibody.

The term “polypeptide” refers to a chain of amino acids of any length, irrespective of modification (e.g. phosphorylation or glycosylation). The term polypeptide includes proteins and fragments thereof. Polypeptides may be “exogenous”, meaning that they are “heterologous”, i.e., foreign to the host cell being utilized, e.g., human polypeptides produced by bacterial cells. Polypeptides are disclosed herein as sequences of amino acid residues. Those sequences are written from left to right in the direction from the amino terminus to the carboxy terminus. According to standard nomenclature, amino acid residue sequences are named in three-letter or one-letter.

The term “amino acids” refers to twenty common naturally occurring amino acids. In some embodiments, the term “amino acid” also includes non-natural amino acids. Any suitable non-natural amino acid may be used. In some embodiments, the non-natural amino acid comprises a reactive portion for affixing the agent to the MIAC.

The term ‘identity’ is defined as the percentage of amino acid residues in a candidate sequence that are identical to amino acid residues in a control polypeptide sequence after comparing the sequences and introducing gaps where necessary to obtain the maximum percentage sequence identity. Comparisons for the purpose of determining percentage amino acid sequence identity can be performed in a variety of ways within the skill of the art, for example, using publicly available computer software, such as BLAST software or the FASTA programme package.

The term ‘at least 80% identity’ means that the percentage of amino acid residues in the candidate sequence that are identical to amino acid residues in the control polypeptide sequence is 80% or more, including 80%, 81%, 82%, 83%, 84%, 85%, 86%, 87%, 88%, 89%, 90%, 91%, 92%, 93%, 94%, 95%, 96%, 97%, 98%, 99%, 100%.

The term “amino acid mutation” refers to a mutation or change in the amino acids of a variant protein or polypeptide as compared to the original protein or polypeptide, including an insertion, deletion or substitution of one or more amino acids in the original protein or polypeptide.

The term “nucleic acid molecule” refers to DNA molecules and RNA molecules. Nucleic acid molecules may be single-stranded or double-stranded, but preferably double-stranded DNA, and nucleic acids are “efficiently linked” when placed in a functional relationship with another nucleic acid sequence.

The term “host cell” refers to a cell that has been or is capable of being transformed with a nucleic acid sequence and thus expressing the selected target gene. The term includes the progeny of the parental cell, whether or not the progeny is morphologically or genetically identical to the original parental cell, as long as the selected target gene is present in the progeny. Commonly used host cells include bacteria, yeast, mammalian cells, etc.

The term “vector” refers to a nucleic acid molecule capable of proliferating another nucleic acid to which it is attached. The term includes vectors that are self-replicating nucleic acid structures and vectors that are incorporated into the genome of the host cell into which they are introduced. Certain vectors are capable of directing the expression of nucleic acids operably linked to them. Such vectors are referred to herein as “expression vectors”.

The term “pharmaceutically acceptable carrier” includes any standard drug carrier, such as phosphate buffered saline solutions, water and emulsions, as well as various types of wetting agents.

The term “positive control” refers to a natural or engineered cell or antibody that binds or expresses a target protein, and a positive control herein refers to a single target positive control.

The term “negative control” refers to a control used in the same experiment to eliminate experimental background effects on experimental values from non-specifically bound samples in the experiment and to provide a better indication of the effect of the experiment by using the same genus, the same subtype, the same dose, the same immunoglobulins and subtypes, and the same markers as the experimental samples.

The term “affinity” refers to the strength of the sum of non-covalent interactions between single binding sites of a molecule (e.g., the antigen-binding module of MIAC) and its binding ligand (e.g., the antigen). Within each antigenic site, the variable region of the antibody “arm” interacts with the antigen at multiple amino acid sites through weak non-covalent forces; the greater the interaction, the stronger the affinity. Unless otherwise indicated, “binding affinity” as used herein refers to the intrinsic binding affinity that reflects a 1:1 interaction between members of a binding pair (e.g., antibody and antigen). The affinity of a molecule X for its ligand Y can generally be expressed as a dissociation constant (Kd). The affinity can be measured by commonly used methods known in the art, such as by using surface plasmon resonance (SPR) techniques (e.g., instrumentation) or biolayer interferometry (e.g., instrumentation).

The term “effector cell” is a leukocyte that expresses one or more FcRs and performs an effector function. In one aspect, the effector cell expresses at least FcγRIII and performs an ADCC effector function. Examples of human leukocytes mediating ADCC include peripheral blood mononuclear cells (PBMC), natural killer (NK) cells, monocytes, cytotoxic T cells, and neutrophilic leukocytes. Effector cells can be isolated from natural sources (e.g., blood). Effector cells are generally lymphocytes associated with the effector phase and are used to produce cytokines (helper T cells), kill cells infected with pathogens (cytotoxic T cells), or secrete antibody (differentiated B cells).

Kabat is the most commonly used and defines CDRs based on sequence variability; Chothia defines CDRs based on sequence variability based on the position of the structural loop region; the IMGT system defines CDRs based on sequence variability and position within the variable domain structure; AbM is based on Oxford Molecular's AbM antibody modelling software and is a compromise between Kabat and Chothia; Contact defines the CDR based on the analysis of complex crystal structures and is similar to Chothia in several respects. Unless otherwise noted, this patent uses Kabat to define CDR.

### BRIEF DESCRIPTION OF THE DRAWINGS

FIG. 1 shows a depiction of exemplary bispecific antibody, wherein a full-length antibody can specifically recognize a first antigen (BAFFR) is fused to a second antigen (BAFFR)-binding portion and a third antigen (CD3)-binding portion, and the second antigen-binding portion is Fab, the second antigen-binding portion is fused to the N-terminus of one heavy chain of the first antigen-binding portion, the third antigen-binding portion is scFv, the third antigen-binding portion substitutes the Fab region of the first antigen-binding portion fused to the second antigen-binding portion, and the bispecific antibody has a knob-Fc in the first Fc region and a hole-Fc in the second Fc region.

FIG. 2 shows a depiction of exemplary bispecific antibody, a full-length antibody can specifically recognize a first antigen (BAFFR) fused to a second antigen (BAFFR)-binding portion, a third antigen (CD3)-binding portion, wherein the second antigen-binding portion is Fab, the second antigen-binding portion is fused to the N-terminus of one heavy chain of the first antigen-binding portion, the third antigen-binding portion is Fab wherein the VH and VL of the third antigen-binding portion are interchanged, the third antigen-binding portion substitutes the Fab region of the first antigen-binding portion fused to the second antigen-binding portion, and the bispecific antibody has a knob-Fc in the first Fc region and a hole-Fc in the second Fc region.

FIG. 3 shows the structure of the bispecific antibody C. The full-length antibody that specifically recognizes the first antigen (BAFFR) is fused to the second antigen (CD3) binding portion, the second antigen-binding portion is Fab, the VH and VL of the second antigen-binding portion are interchanged, the second antigen-binding portion substitutes the Fab region on the side of the first antigen-binding portion, the first Fc region of the bispecific antibody is knob-Fc and the second Fc region is hole-Fc.

FIG. 4 shows the binding activity of the bispecific antibody to BAFFR protein.

FIG. 5 shows the binding activity of the bispecific antibody to CD3 protein.

FIG. 6 shows the binding activity of the bispecific antibody to Jeko-1 cells.

FIG. 7 shows the binding activity of the bispecific antibody to Raji cells.

FIG. 8 shows the binding activity of the bispecific antibody to Su-DHL4 cells.

FIG. 9 shows the T cell-specific activation activity of the bispecific antibody to jukart-NFAT-luc reporter gene cells.

FIG. 10 shows the killing activity of the bispecific antibody to CHO-K1-BAFFR cells.

FIG. 11 shows the killing activity of the bispecific antibody to CHO-K1 cells.

FIG. 12 shows the killing activity of the bispecific antibody to Raji-luc cells.

### DETAILED DESCRIPTION

The present invention is further described below in connection with the accompanying drawings and specific embodiments, the protection of which is not limited to the following embodiments. It should also be understood that the terms used in embodiments of the present invention are intended to describe particular specific embodiments and are not intended to limit the scope of protection of the present invention. Without departing from the spirit and scope of the inventive concept, variations and advantages that can be envisaged by those skilled in the art are included in the present invention and the scope of protection of the invention by the appended claims and any equivalents thereof.

### Example 1 Acquisition and Optimization of Nucleotide Sequences

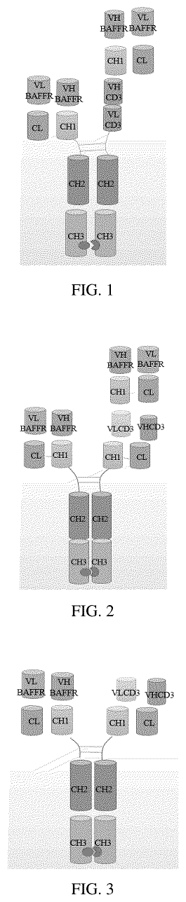

The embodiment is to construct bispecific antibody for BAFFR and CD3 targets according to FIGS. 1-3, named antibody A, antibody B, and antibody C, in order.

The Fc of the antibody amino acid sequence was adjusted to other IgG types as needed, and further amino acid mutations of the desired form were designed in each heavy chain, from which the amino acid sequence of the target antibody was obtained (see Table 1), and the amino acid sequence combinations of the constructed antibodies are shown in Table 2 and include theoretical molecular weights.

TABLE 1
Antibody sequence information

| Peptide Chain | Amino Acid Sequence |
|---|---|
| VH(BAFFR) | SEQ ID NO: 1 |
| VL(BAFFR) | SEQ ID NO: 2 |
| VH(CD3) | SEQ ID NO: 9 |
| VL(CD3) | SEQ ID NO: 10 |
| CL | SEQ ID NO: 17 |
| Constant Region Sequence of IgG4 | SEQ ID NO: 18 |
| Heavy Chain (S228P, Y407A) | |
| Constant Region Sequence of IgG4 | SEQ ID NO: 19 |
| Heavy Chain (S228P, T366W) | |
| Linker | SEQ ID NO: 20 |

TABLE 2
Sequence combinations of antibody

| | Light | Heavy | Heavy | Light | Light |
|---|---|---|---|---|---|
| Antibody | Chain 1 | Chain 1 | Chain 2 | Chain 2 | Chain 3 |
| Antibody A | SEQ ID | SEQ ID | SEQ ID | SEQ ID | / |
| | NO: 21 | NO: 22 | NO: 23 | NO: 21 | |
| Antibody B | SEQ ID | SEQ ID | SEQ ID | SEQ ID | SEQ ID |
| | NO: 21 | NO: 22 | NO: 24 | NO: 25 | NO: 21 |
| Antibody C | SEQ ID | SEQ ID | SEQ ID | SEQ ID | / |
| | NO: 21 | NO: 22 | NO: 26 | NO: 25 | |

The above target amino acid sequences were converted into nucleotide sequences and optimized for a series of parameters that may affect the expression of the antibody in mammalian cells: Codon preference, GC content (i.e., the ratio of guanine G to cytosine C among the four bases of DNA), CpG islands (i.e., regions of the genome where CpG dinucleotides are in high density), secondary structure of mRNA, splice sites, pre-mature PolyA sites, internal Chi sites (a short segment of DNA in the genome with an increased chance of homologous recombination near the site) or ribosome binding sites, RNA destabilizing sequences, reverse repetitive sequences, and restriction enzymes that may interfere with cloning; In addition, related sequences such as Kozak sequences and SD sequences, which may improve translation efficiency, were added. Heavy chain and light chain genes encoding the above antibodies were designed, and nucleotide sequences encoding signal peptides optimized according to amino acid sequences were designed at the 5′ end of the heavy and light chains, respectively; in addition, stop codons were added to the 3′ end of the light and heavy chain nucleotide sequences, respectively.

### Example 2 Gene Synthesis and Construction of Expression Vectors

The pcDNA3.1-G418 vector was used as a plasmid vector for expressing the multifunctional antibody. The pcDNA3.1-G418 vector comprises the promoter CMVPromoter, the eukaryotic screening marker G418 tag and the prokaryotic screening tag Ampicilline. The nucleotide sequences of the light and heavy chains of the constructed antibody expression were obtained by gene synthesis. The vector and the target fragment were double digested by HindIII and XhoI. After recovery, the vector was ligated by DNA ligase, and transformed into Escherichia coli competent cell DH5 α. The positive clones were selected and subjected to plasmid extraction and enzyme digestion verification to obtain the antibody containing plasmid.

### Example 3 Plasmid Extraction

The recombinant plasmids containing each of the above target genes were transformed into E. coli receptor cells DH5a, the transformed bacteria were coated on LB plates containing 100 μg/mL ampicillin, the plasmid clones were selected and cultured in liquid LB medium, shaken at 260 rpm for 14 h. The plasmids were extracted by an endotoxin-free plasmid macroextraction kit, lysed in sterile water and the concentrations were determined using a nucleic acid protein quantifier.

### Example 4 Plasmid Transfection, Transient Expression and Antibody Purification

The ExpiCHO was cultured at 37 °C, with 8% CO₂, at 100 rpm to a cell density of 6×10⁶ cells/mL. The constructed plasmids were transfected into the above cells according to the combination pairing using liposomes at a concentration of 1 mg/mL, determined by reference to the ExpiCHO™ Expression System kit. The cells were incubated at 32 °C, with 5% CO₂, and 100 rpm for 7-10 days. The product was replenished once after 18-22 h of transfection and again between day 5. The above culture products were centrifuged at 4000 g, and the medium supernatant was filtered through a 0.22 μm filter membrane and collected. The resulting antibody proteins were purified using ProteinA, ionic columns and the eluate was collected.

ProteinA, ionic column purification is performed as follows: cell culture broth is centrifuged at high speed and the supernatant is taken for affinity chromatography using GE's ProteinA chromatography column. The chromatography was performed using an equilibration buffer of 1× PBS (pH 7.4). The cell supernatant was combined and washed with PBS until UV was returned to baseline, then the target protein was eluted using an elution buffer of 0.1 M glycine (pH 3.0) and stored at a neutral pH using Tris. The affinity chromatography product is adjusted to a pH 1-2 pH units below or above the isoelectric point pI and diluted appropriately to control the sample conductance below 5 ms/cm. A NaCl gradient elution at the corresponding pH is carried out using a suitable corresponding pH buffer such as phosphate buffer, acetate buffer, etc., using conventional ion exchange chromatography methods in the art such as anion exchange or cation exchange, and the collection tubes where the target proteins are located are combined and stored according to SDS-PAGE.

Then, the purified eluate is ultrafiltered into a buffer. Proteins were detected by SDS-polyacrylamide gel electrophoresis assay.

It was proved by SDS-PAGE that the non-reducing gel condition contains the target bands, and the target antibodies under the reducing gel all contain the target bands, corresponding to the heavy chain and the light chain of the desired antibody. Therefore, the structurally correct antibodies were proved to be obtained after transfection, transient expression and purification of the described plasmids.

### Example 5 Detection of Affinity of Antibody to BAFFR by ELISA

Human-BAFFR-His (purchased from Acro, Cat: BAR-H52H3) was diluted to 1 μg/mL using PBS buffer (pH 7.4) and added to a 96-well ELISA plate at 100 μL per well, and coated overnight at 4 °C. After 1 hour closure with 1% BSA closure solution, the plate was washed three times with PBST, and the constructed antibody was diluted to 100 nM with 0.5% BSA sample diluent as the starting concentration and diluted in a 3 fold gradient for a total of 11 gradients, and was incubated at 100 μL per well for 1 h at 37 °C. The plate was then washed 3 times with PBST and HRP-labelled goat anti-human IgG-Fc (purchased from Jackson, Cat: 109-035-098) was diluted 1:20,000 with sample diluent. 100 μL was added to each well and incubated for 1 h at room temperature. Negative control (irrelevant antibody) and positive control were set, the positive control was BAFFR monoclonal antibody (BAFFR monoclonal antibody sequence consists of 1 and 2, which added the constant region of human IgG1, see 27 and 28). After washing the plate 4 times with PBST, 100 μL of TMB substrate was added to each well, and incubation was performed at room temperature and protected from light for 10 minutes. The color development reaction was terminated by adding 100 μL of 1M HCl solution to each well. Readings were taken on a multifunctional enzyme marker.

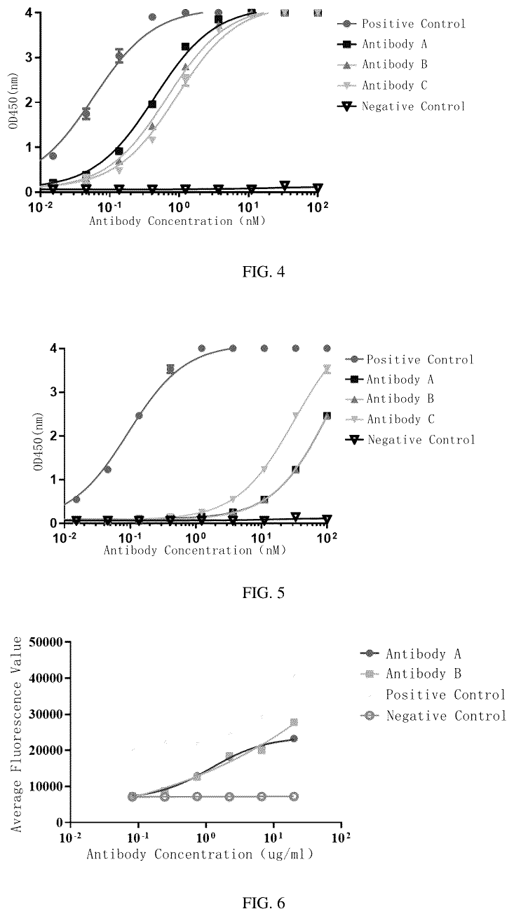

The ELISA results of the antibody are shown in FIG. 4, and the bispecific antibody can bind to the BAFFR protein at all concentrations.

### Example 6 Detection of Affinity of Antibody to CD3 by ELISA

Human-CD3-His (purchased from Acro, Cat: CDD-H52W4) was diluted to 0.2 μg/mL using PBS buffer (pH 7.4) and added to a 96-well ELISA plate at 100 μL per well, and coated overnight at 4 °C. After 1 hour closure with 1% BSA closure solution, the plate was washed three times with PBST, and the purified antibody was diluted to 100 nM with 0.5% BSA sample diluent as the starting concentration and diluted in a 3 fold gradient for a total of 11 gradients, with a negative control (blank well and IgG1 isotype control) and a positive control, which was CD3 monoclonal antibody (the sequence of CD3 antibody was acquired from INN blinatumomab, the sequence consisted of SEQ ID NO: 9, SEQ ID NO: 10, which was added to the constant region of human IgG1, the constant region of IgG1 is shown in SEQ ID NO: 27 and SEQ ID NO: 28), and was incubated at 100 μL per well for 1 h at 37 °C. The plate was then washed 3 times with PBST and HRP-labelled goat anti-human IgG-Fc was diluted 1:20,000 with sample diluent, 100 μL was added to each well and incubated for 1 h at room temperature. After washing the plate 4 times with PBST, 100 μL of TMB substrate was added to each well and incubated for 10 min at room temperature and protected from light. 100 μL of 1M HCl was added to each well to terminate the colour development reaction. The absorbance value of each well in the 96-well plate was measured on a multifunctional enzyme marker at a wavelength of 450 nm and a reference wavelength of 570 nm

The ELISA results of the antibody are shown in FIG. 5, and the bispecific antibody can bind to CD3 at all concentrations.

### Example 7 Detection of the Binding Activity of Antibody to BAFFR Positive Tumor Cells by Flow Cytometry

BAFFR positive tumor cells Jeko-1, Raji, Su-DHL4 (from CAS Cell Preservation Center) with logarithmic growth phase and normal morphology were taken, transferred to centrifuge tube and centrifuged at 1000 rpm for 5 min, and after resuspension of the cells with diluent, 3×10⁵/well was taken and added into 96-well cell culture plate. The antibody obtained from purification was diluted to 20 μg/mL with FACS buffer, and used as the starting concentration for 3-fold gradient dilution, with a total of 6 gradients, and set up irrelevant antibody negative control and positive control, the positive control was BAFFR monoclonal antibody (BAFFR monoclonal antibody sequence consisting of SEQ ID NO: 1, SEQ ID NO: 2, which was added to the constant region of human IgG1, the constant region of human IgG1 is shown in SEQ ID NO: 27 and SEQ ID NO: 28), and 100 μL antibody dilution was added. Cells were incubated at 4 °C for 60 min and then washed twice with excess FACS buffer. Cells were resuspended in 100 μL of FACS buffer and fluorescent secondary antibody-FITC (Biolegend, Cat: 109306) against human IgG Fc was added to the samples, incubated for 30 min and washed twice with excess FACS buffer. Cells were resuspended in flow buffer and subsequently detected and analyzed by flow cytometry.

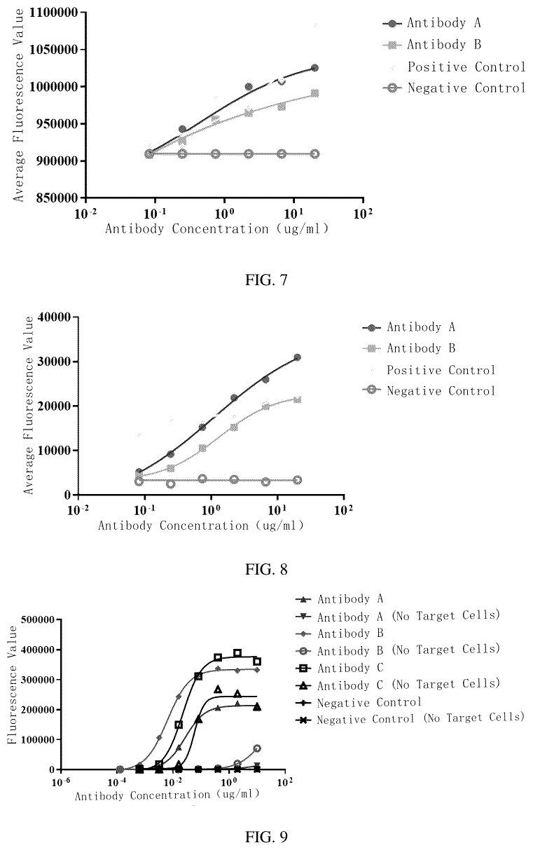

The results of FACs for cell binding activity of the antibody to BAFFR-positive tumor cells Jeko-1, Raji, and SuDHL4 are shown in FIGS. 6-8, where both bispecific antibody A and antibody B can bind specifically to BAFFR-positive tumor cells at a wide range of concentrations.

### Example 8 Specific Activation of T Cells by Constructed Antibody

BAFFR-positive Jeko-1 cells (from CAS Cell Conservation Center) were used to spread in 96-well plates at 2×10⁴/well, and then the effector cells, Jurkat-NFAT-Luc, were added at 5×10⁴/well. The antibody was diluted to a starting concentration of 30 μg/mL using PBS, and 5-fold dilutions were made, with a total of 9 concentration gradients, and added to the wells of the corresponding cells. Negative irrelevant controls were set and incubated at 37 °C for 6 h. And then Bio-Lite was added to the sample wells and incubated at room temperature for 10 min and then read by multifunctional enzyme marker.

As shown in FIG. 9, the experimental results indicate that bispecific antibody A and antibody B specifically activate CD3-expressing jurkart-NFAT-Luc reporter gene cells in the presence of BARRF-positive target cells Jeko-1. In contrast, there was no activation activity in the absence of antibody or in the presence of no target cells. It is shown that recognition of BAFFR-positive cells by bispecific antibody A and antibody B can specifically activate CD3-positive T cells, which in turn activate downstream signals inducing tumor killing by T cells. There is a risk of non-specific activation of the bispecific antibody C.

### Example 9 Specific Killing of CHO-K1-BAFFR Cells by Constructed Antibody

BAFFR-positive CHO-K1-BAFFR cells (an engineered cell line overexpressing BAFFR, constructed by lentiviral transduction of CHO-K1 cells) and BAFFR-negative CHO-K1 cells were seeded at 2×10⁴ cells/well in 96-well plates. After 24 h of incubation, bispecific and irrelevant antibody were added, with 10 μg/mL start, followed by 10-fold dilution, for a total of 6 concentration gradients, meanwhile, CIK (CD3+CD56+ cells) effector cells were added at 1×10⁵/well, with a effector-to-target (E:T) ratio of 5:1. A blank control (dilution), a negative control (target cells+CIK cells, no antibody), and an irrelevant antibody group were set. Negative control 1 (BAFFR monoclonal antibody sequence consisting of SEQ ID NO: 1, SEQ ID NO: 2, and adding the constant region of human IgG1, which is shown in SEQ ID NO: 27 and SEQ ID NO: 28) was BAFFR monoclonal antibody and negative control 2 was anti-GPC3 and CD3 antibody (Antibody from patent U.S. Pat. No. 11,001,643B2, amino acid sequence consisting of SEQ ID NO: 29, SEQ ID NO: 30 and SEQ ID NO: 31) in the negative control group. After 24 h incubation in the cell culture incubator, it was wetted with PBS for 3 times. 100 μL of medium containing 10% CCK-8 (Cat: CK04) was added, incubated in the incubator for 3 h, and detected at 450 nm OD by enzyme marker. The cell killing rate was calculated with the following formula:

Cell killing rate (%)=(1−(OD of sample well−OD of blank)/(OD of negative well−OD of blank))×100%.

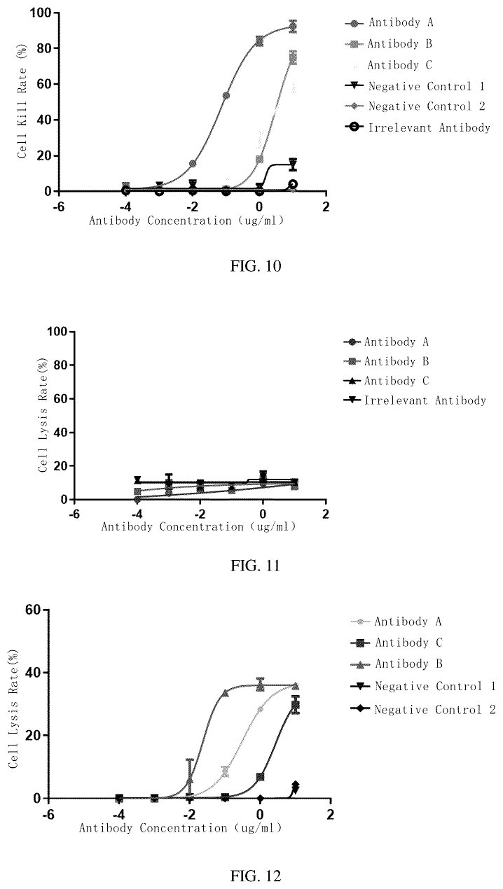

As can be seen from FIGS. 10-11, the bispecific antibody can kill BAFFR overexpressed CHO-K1-BAFFR, and the irrelevant antibody does not have a killing effect, whereas both the bispecific antibody and the irrelevant antibody do not have a killing effect on CHO-K1, suggesting that the bispecific antibody can mediate the specific killing of BAFFR-positive cells by CIK cells.

### Example 10 Specific Killing of Raji-Luc Cells by Constructed Antibody

BAFFR-positive Raji-luc (made by transducing raji with Luc lentivirus) were seeded at 1×10⁴ cells/well in 96-well plates. After 24 h of incubation, bispecific and irrelevant antibody were added, with 50 μg/mL start, followed by 10-fold dilution, for a total of 6 concentration gradients. Meanwhile, CIK (CD3+CD56+ cells) effector cells were added at 1×10⁵/well, with a effector-to-target (E:T) ratio of 10:1. A blank control (dilution), a negative control (target cells+CIK cells, no antibody), and an irrelevant antibody group were set. Negative control 1 (BAFFR monoclonal antibody sequence consisting of SEQ ID NO: 1, SEQ ID NO: 2, and adding the constant region of human IgG1, which is shown in SEQ ID NO: 27 and SEQ ID NO: 28) was BAFFR monoclonal antibody and negative control 2 was anti-GPC3 and CD3 antibody (Antibody from patent U.S. Pat. No. 11,001,643B2, amino acid sequence consisting of SEQ ID NO: 29, SEQ ID NO: 30 and SEQ ID NO: 31) in the negative control group. After 24 h incubation in the cell culture incubator, Bio-Lite was added, incubated in the incubator for 10 min, and detected at 450 nm OD by enzyme marker. The cell killing rate was calculated with the following formula:

Cell killing rate (%)=(1−(OD of sample well−OD of blank)/(OD of negative well−OD of blank))×100%.

As can be seen in FIG. 12, the bispecific antibody can kill BAFFR-positive Raji, and the irrelevant antibody does not have a killing effect, suggesting that the bispecific antibody mediates the specific killing of BAFFR-positive cells by CIK cells.

The scope of protection of the present invention is not limited to the above embodiments. Without departing from the spirit and scope of the inventive conception, variations and advantages that can be thought of by those skilled in the art are included in the present invention and are protected by the appended claims.

1. A bispecific antibody, comprising:
(a) a first antigen-binding portion that specifically binds to the first antigen, wherein the first antigen is BAFFR;
(b) a second antigen-binding portion that specifically binds to the second antigen, wherein the second antigen is BAFFR; and
(c) a third antigen-binding portion that specifically binds to the third antigen, wherein the third antigen is CD3.

2. The bispecific antibody of claim 1, wherein the first antigen-binding portion is a full-length antibody consisting of two heavy chains and two light chains.

3. The bispecific antibody of claim 1, wherein the second antigen-binding portion and/or the third antigen-binding portion is an antibody fragment comprising a heavy chain variable domain (VH) and/or a light chain variable domain (VL).

4. The bispecific antibody of claim 3, wherein the second antigen-binding portion and/or the third antigen-binding portion is Fab, Fab′, scFab, F(ab′)2, Fv, dsFv, or scFv.

5. The bispecific antibody of claim 1, wherein the second antigen-binding portion is fused to the N-terminus and/or C-terminus of one heavy chain or both heavy chains of the first antigen-binding portion.

6. The bispecific antibody of claim 1, wherein the second antigen-binding portion is fused to the N-terminus and/or C-terminus of one light chain or both light chains of the first antigen-binding portion.

7. The bispecific antibody of claim 5, wherein the third antigen-binding portion substitutes one or two of Fab regions or Fv regions of the first antigen-binding portion.

8. The bispecific antibody of claim 1, comprising a first Fc region and a second Fc region.

9. The bispecific antibody of claim 8, wherein the first Fc region and the second Fc region are the same or different.

10.-12. (canceled)

13. The bispecific antibody of claim 8, wherein the first Fc region is knob-Fc and the second Fc region is hole-Fc.

14. The bispecific antibody of claim 8, wherein the first Fc region is hole-Fc and the second Fc region is knob-Fc.

15. The bispecific antibody of claim 1, wherein the VH and VL of the third antigen-binding portion are interchanged.

16.-18. (canceled)

19. The bispecific antibody of claim 1, wherein the first antigen-binding portion specifically binds to BAFFR, wherein HCDR1 of the first antigen-binding portion is shown in SEQ ID NO: 3, or is a sequence having at least 80% identity to SEQ ID NO: 3; HCDR2 is shown in SEQ ID NO: 4, or is a sequence having at least 80% identity to SEQ ID NO: 4; HCDR3 is shown in SEQ ID NO: 5, or is a sequence having at least 80% identity to SEQ ID NO: 5; LCDR1 is shown in SEQ ID NO: 6, or is a sequence having at least 80% identity to SEQ ID NO: 6; LCDR2 is shown in SEQ ID NO: 7, or is a sequence having at least 80% identity to SEQ ID NO: 7; and LCDR3 is shown in SEQ ID NO: 8, or is a sequence having at least 80% identity to SEQ ID NO: 8.

20. The bispecific antibody of claim 1, wherein the second antigen-binding portion specifically binds to BAFFR, wherein HCDR1 of the second antigen-binding portion is shown in SEQ ID NO: 3, or is a sequence having at least 80% identity to SEQ ID NO: 3; HCDR2 is shown in SEQ ID NO: 4, or is a sequence having at least 80% identity to SEQ ID NO: 4; HCDR3 is shown in SEQ ID NO: 5, or is a sequence having at least 80% identity to SEQ ID NO: 5; LCDR1 is shown in SEQ ID NO: 6, or is a sequence having at least 80% identity to SEQ ID NO: 6; LCDR2 is shown in SEQ ID NO: 7, or is a sequence having at least 80% identity to SEQ ID NO: 7; and LCDR3 is shown in SEQ ID NO: 8, or is a sequence having at least 80% identity to SEQ ID NO: 8.

21. The bispecific antibody of claim 1, wherein the third antigen-binding portion specifically binds to CD3, wherein HCDR1 of the third antigen-binding portion is shown in SEQ ID NO: 11, or is a sequence having at least 80% identity to SEQ ID NO: 11; HCDR2 is shown in SEQ ID NO: 12, or is a sequence having at least 80% identity to SEQ ID NO: 12; HCDR3 is shown in SEQ ID NO: 13, or is a sequence having at least 80% identity to SEQ ID NO: 13; LCDR1 is shown in SEQ ID NO: 14, or is a sequence having at least 80% identity to SEQ ID NO: 14; LCDR2 is shown in SEQ ID NO: 15, or is a sequence having at least 80% identity to SEQ ID NO: 15; and LCDR3 is shown in SEQ ID NO: 16, or is a sequence having at least 80% identity to SEQ ID NO: 16.

22. The bispecific antibody of claim 19, wherein the first antigen-binding portion specifically binds to BAFFR, wherein the heavy chain variable region VH of the first antigen-binding portion is as shown in SEQ ID NO: 1, or is a sequence having at least 80% identity to SEQ ID NO: 1; and the light chain variable region VL is as shown in SEQ ID NO: 2, or is a sequence having at least 80% identity to SEQ ID NO: 2.

23. The bispecific antibody of claim 19, wherein the second antigen-binding portion specifically binds to BAFFR, wherein the heavy chain variable region VH of the second antigen-binding portion is as shown in SEQ ID NO: 1, or is a sequence having at least 80% identity to SEQ ID NO: 1; and the light chain variable region VL is as shown in SEQ ID NO: 2, or is a sequence having at least 80% identity to SEQ ID NO: 2.

24. The bispecific antibody of claim 19, wherein the third antigen-binding portion specifically binds to CD3, wherein the heavy chain variable region VH of the third antigen-binding portion is as shown in SEQ ID NO: 9, or is a sequence having at least 80% identity to SEQ ID NO: 9; and the light chain variable region VL is as shown in SEQ ID NO: 10, or is a sequence having at least 80% identity to SEQ ID NO: 10.

25.-30. (canceled)

31. A pharmaceutical composition, comprising the bispecific antibody of claim 1 and a pharmaceutically acceptable carrier, diluent, or excipient.

32. An antibody drug conjugate, comprising the bispecific antibody of claim 1.

33. (canceled)
